(19) United States
(12) Patent Application Publication
(10) Pub. No.: US 2008/0043910 A1
(43) Pub. Date: Feb. 21, 2008

(54) METHOD AND APPARATUS FOR STABILIZING AN ENERGY SOURCE IN A RADIATION DELIVERY DEVICE

(75) Inventor: Mark Thomas, Madison, WI (US)

Correspondence Address: MICHAEL BEST & FRIEDRICH LLP, 100 E WISCONSIN AVENUE, Suite 3300, MILWAUKEE, WI, 53202, US

(73) Assignee: TOMOTHERAPY INCORPORATED, Madison, WI (US)

(21) Appl. No.: 11/838,790

(22) Filed: Aug. 14, 2007

Related U.S. Application Data
(60) Provisional application No. 60/837,901, filed on Aug. 15, 2006.

(51) Int. Cl. A61N 5/10; H05H 9/00

(52) U.S. Cl. 378/65; 315/505; 607/101

(57) ABSTRACT

A radiation delivery device and method of stabilizing a microwave energy source. The device includes a microwave energy source, and a microwave utilization device coupled to the energy source. A non-reciprocal transmission device couples the source to the utilization device, the transmission device receiving an unutilized portion of the energy from the device. The transmission device conditions the unutilized energy and returns the conditioned energy to the source. The transmission device comprises a first component that operates to adjust a first property of the energy such that the adjustment does not affect any other properties of the energy. The returned conditioned energy functions to modify the frequency of the source such that the unutilized energy in the system is minimized, thereby stabilizing the frequency of the energy output by the source.

### REFERENCE TO RELATED APPLICATIONS

This application claims priority to provisional patent application No. 60/837,901, filed Aug. 15, 2006, the entire contents of which is incorporated by reference herein.

### FIELD OF THE INVENTION

This invention relates to a radiation delivery device, such as a radiation therapy treatment system. More specifically, the invention relates to a method of and apparatus for stabilizing the operating frequency of a microwave energy source, such as a magnetron oscillator, to optimize output from a particle accelerator within the system.

### BACKGROUND

Medical equipment for radiation therapy treats tumorous tissue with high energy radiation. The dose and the placement of the dose must be accurately controlled to insure both that the tumor receives sufficient radiation to be destroyed, and that damage to the surrounding and adjacent non-tumorous tissue is minimized. Intensity modulated radiation therapy (IMRT) treats a patient with multiple rays of radiation each of which may be independently controlled in intensity and/or energy. The rays are directed from different angles about the patient and combine to provide a desired dose pattern. In external source radiation therapy, a radiation source external to the patient treats internal tumors. The external source is normally collimated to direct a beam only to the tumorous site. Typically, the radiation source consists of either high-energy X-rays, electrons, or gamma rays from highly collimated radioisotopes. The radiation source that performs this type of radiation therapy can include a device known as a magnetron, which provides microwave power to a linear accelerator (“LINAC”).

### SUMMARY

To effectively treat a patient with IMRT using a magnetron-driven linear accelerator, the dose output from the LINAC needs to be stable. The magnetron provides a compact power source that is amenable to use with a modest-sized rotating radiation therapy delivery system, such as the system that will be described in detail below. However, the linear accelerator generally requires greater frequency stability for constant output than can readily be achieved by a magnetron. Mechanical vibration of the magnetron can cause sufficient frequency variation to produce greater output variation than is desirable in a continuous-delivery IMRT system. Thus, the invention provides a means to stabilize the magnetron frequency to achieve the desired constant output. In effect, the invention is directed to taking a source device, such as a magnetron, having broad characteristics and constraining the device to operate within a narrower bandwidth due to coupling the device to a highly resonant load (e.g., the LINAC). To prevent instability in the system, the output frequency of the magnetron should match the operating frequency of the LINAC.

In one embodiment, the invention provides a radiation delivery device. The device includes a microwave energy source, such as a magnetron, and a microwave utilization device coupled to the energy source. A non-reciprocal transmission device couples the source to the utilization device and receives an unutilized portion of the energy from the utilization device. The transmission device conditions at least a portion of the unutilized energy and returns the conditioned energy to the energy source. The transmission device includes a first component that operates to adjust a first property of the energy such that the adjustment does not affect any other properties of the energy. The returned conditioned energy functions to modify the frequency of the energy source such that the unutilized energy in the radiation delivery device is minimized.

In this case, the utilization device, which in the illustrated embodiment is a particle accelerator, has an operational bandwidth that is narrower than that inherent in the energy generation source. The difference is due to the need for the magnetron to gather electrons from a large fraction of the phase space occupied by the thermal spread of its high current electron beam in order to achieve high efficiency conversion of the beam energy to radio frequency (RF) oscillation. The accelerator's narrow bandwidth is a consequence of the goal of achieving maximized internal electric fields in order to produce the highest radiant energies in the shortest practical length for a given power input. The method of stabilizing the system according to the invention can be applied to any RF-accelerated particle/photon based modality.

Microwave energy generated by the magnetron is passed through a non-reciprocal transmission device, such as a waveguide, to a linear accelerator, which utilizes the energy to generate high energy electrons and/or X-rays. The output frequency of the magnetron is mechanically tuned to the resonant operating frequency of the LINAC, using a conventional automatic frequency control feedback loop to track thermal variations. Microwave energy is reflected from the LINAC when the frequency of the energy from the magnetron does not match that of the LINAC's resonant operating frequency. The reflected energy is routed through the circulator to waveguide components that separately control the amplitude and phase of the reflected energy. The amplitude and phase controlled energy then returns to the magnetron where the controlled energy exerts a frequency pulling effect. When the amplitude and phase of the reflected energy are applied in a specific manner, the pulling effect is in opposition to small deviations of the magnetron frequency from the resonant frequency of the LINAC. This effect occurs within a fraction of a microsecond, in contrast to the several millisecond response time of a mechanical tuning system.

The invention also provides a method of stabilizing a microwave energy source, the source being coupled to a microwave utilization device. The method includes coupling a non-reciprocal transmission device to the source, and directing energy from the source through the transmission device to the utilization device. At least a portion of the energy returned from the utilization device is conditioned, wherein conditioning includes modifying a first characteristic of the energy without affecting any other characteristics of the energy. The conditioned portion of the energy is returned to the energy source, and functions to stabilize the frequency output of the energy source.

Other aspects of the invention will become apparent by consideration of the detailed description and accompanying drawings.

### BRIEF DESCRIPTION OF THE DRAWINGS

FIG. 1 is a perspective view of a radiation therapy treatment system.

FIG. 2 is a perspective view of a multi-leaf collimator that can be used in the radiation therapy treatment system illustrated in FIG. 1.

FIG. 3 is a front view of a magnetron for use with the system of FIG. 1.

FIG. 4 is a schematic view of the RF subsystem for use with the system of FIG. 1.

FIG. 5 is a block diagram of the radiation module of the system of FIG. 1, schematically represented as a circuit.

FIG. 6 is a block diagram of an alternate embodiment of the invention, utilizing a 3-port circulator, schematically represented as a circuit.

FIG. 7 is a Rieke diagram illustrating the dependencies of frequency and power output on the load impedance of the system of FIG. 4.

FIG. 8 is a graphical representation of the input impedance of the LINAC and of frequency lines from the magnetron Rieke diagram of FIG. 7, superimposed in the complex impedance plane.

FIG. 9 is a graphical representation of how adjustment of the magnetron moves the Rieke diagram frequency lines in the complex impedance plane.

FIG. 10 is a graphical representation of how the LINAC's impedance contour varies with the magnitude of reflection coefficient at port 3 of a 4-port circulator for use in the system of FIG. 4.

FIG. 11 is a graphical representation of how the LINAC's impedance contour varies with the phase of a unit reflection coefficient at port 4 of the 4-port circulator.

FIG. 12 is a graphical representation of how the upper limit of reflected power that can be returned to the magnetron is constrained by the choice of the fractional reflection coefficient at port 3 of the 4-port circulator.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Although directional references, such as upper, lower, downward, upward, rearward, bottom, front, rear, etc., may be made herein in describing the drawings, these references are made relative to the drawings (as normally viewed) for convenience. These directions are not intended to be taken literally or limit the present invention in any form. In addition, terms such as “first”, “second”, and “third” are used herein for purposes of description and are not intended to indicate or imply relative importance or significance.

In addition, it should be understood that embodiments of the invention include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic based aspects of the invention may be implemented in software. As such, it should be noted that a plurality of hardware and software based devices, as well as a plurality of different structural components may be utilized to implement the invention. Furthermore, and as described in subsequent paragraphs, the specific mechanical configurations illustrated in the drawings are intended to exemplify embodiments of the invention and that other alternative mechanical configurations are possible.

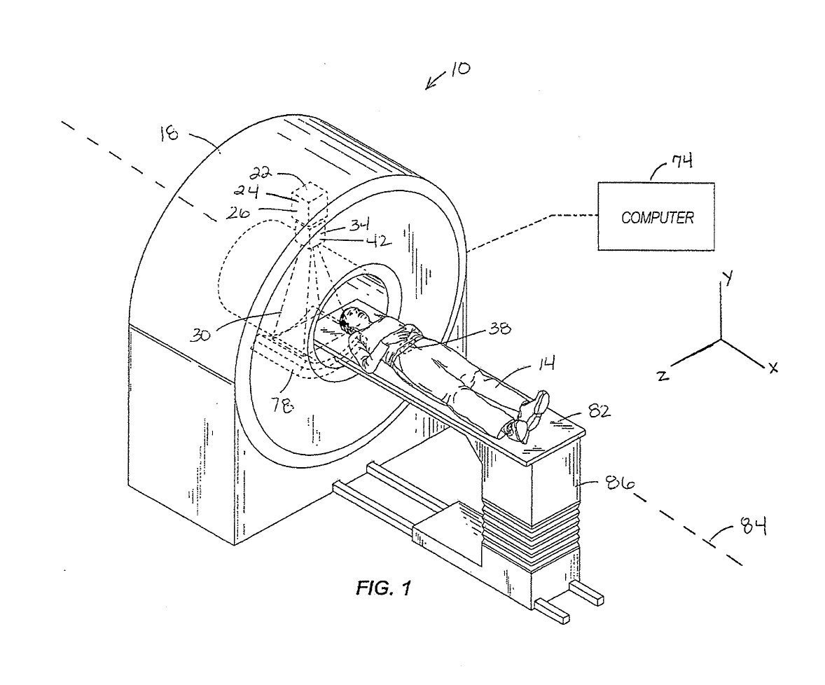

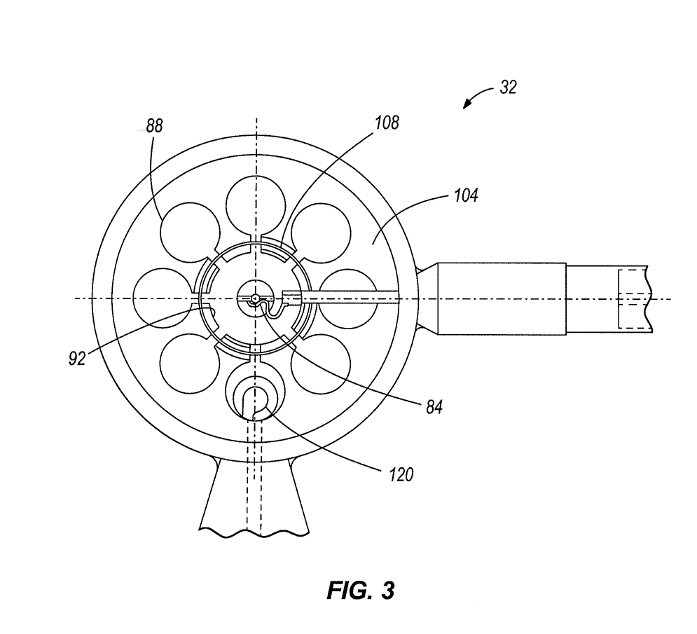

FIG. 1 illustrates a radiation therapy treatment system 10 that can provide radiation therapy to a patient 14. A radiation therapy treatment system such as will be described below is one example of a radiation delivery device that could be operated according to the present invention. The radiation therapy treatment can include photon-based radiation therapy, brachytherapy, electron beam therapy, proton, neutron, or particle therapy, or other types of treatment therapy. The radiation therapy treatment system 10 includes a gantry 18. The gantry 18 can support a radiation delivery module 22, which can include a radiation source 24 and a linear accelerator 26 operable to generate a beam 30 of radiation. Though the gantry 18 shown in the drawings is a ring gantry, i.e., it extends through a full 360° arc to create a complete ring or circle, other types of mounting arrangements may also be employed. For example, a C-type, partial ring gantry, or robotic arm could be used. Any other framework capable of positioning the radiation delivery module 22 at various rotational and/or axial positions relative to the patient 14 may also be employed. In addition, the radiation source 24 may travel in path that does not follow the shape of the gantry 18. For example, the radiation source 24 may travel in a non-circular path even though the illustrated gantry 18 is generally circular-shaped. The radiation source 24 can include an energy generation source, such as a magnetron 32 (illustrated in FIG. 3) that generates energy to be passed to the LINAC 26 (illustrated in FIG. 4). The magnetron 32 and LINAC 26 will be discussed in more detail below.

The radiation module 22 can also include a modulation device 34 operable to modify or modulate the radiation beam 30. The modulation device 34 provides the modulation of the radiation beam 30 and directs the radiation beam 30 toward the patient 14. Specifically, the radiation beam 30 is directed toward a portion of the patient. Broadly speaking, the portion may include the entire body, but is generally smaller than the entire body and can be defined by a two-dimensional area and/or a three-dimensional volume. A portion or area desired to receive the radiation, which may be referred to as a target or target region (shown as 38), is an example of a region of interest. Another type of region of interest is a region at risk. If a portion includes a region at risk, the radiation beam is preferably diverted from the region at risk. Such modulation is sometimes referred to as intensity modulated radiation therapy (“IMRT”).

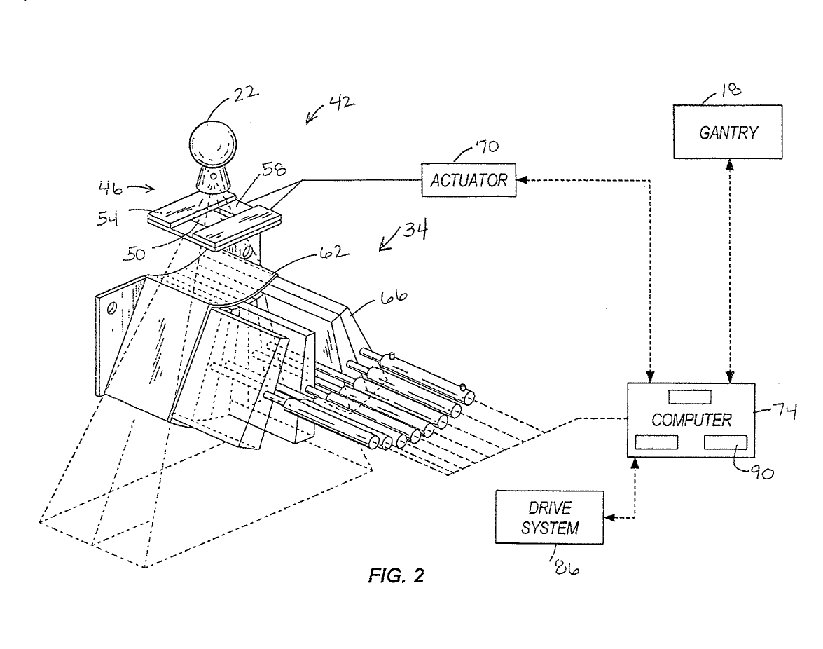

The modulation device 34 can include a collimation device 42 as illustrated in FIG. 2. The collimation device 42 includes a set of jaws 46 that define and adjust the size of an aperture 50 through which the radiation beam 30 may pass. The jaws 46 include an upper jaw 54 and a lower jaw 58. The upper jaw 54 and the lower jaw 58 are moveable to adjust the size of the aperture 50.

In one embodiment, as illustrated in FIG. 2, the modulation device 34 can comprise a multi-leaf collimator 62, which includes a plurality of interlaced leaves 66 operable to move from position to position, to provide intensity modulation. It is also noted that the leaves 66 can be moved to a position anywhere between a minimally and maximally-open position. The plurality of interlaced leaves 66 modulate the strength, size, and shape of the radiation beam 30 before the radiation beam 30 reaches the area 38 on the patient 14. Each of the leaves 66 is independently controlled by an actuator 70, such as a motor or an air valve so that the leaf 66 can open and close quickly to permit or block the passage of radiation. The actuators 70 can be controlled by a computer 74 and/or controller.

The radiation therapy treatment system 10 can also include a detector 78, e.g., a kilovoltage or a megavoltage detector, operable to receive the radiation beam 30, as illustrated in FIG. 1. The LINAC 26 and the detector 78 can also operate as a computed tomography (CT) system to generate CT images of the patient 14. The LINAC 26 emits the radiation beam 30 toward the area 38 in the patient 14. The area 38 absorbs some of the radiation. The detector 78 detects or measures the amount of radiation absorbed by the area 38. The detector 78 collects the absorption data from different angles as the LINAC 26 rotates around and emits radiation toward the patient 14. The collected absorption data is transmitted to the computer 74 to process the absorption data and to generate images of the patient's body tissues and organs. The images can also illustrate bone, soft tissues, and blood vessels.

The radiation therapy treatment system 10 can also include a patient support, such as a couch 82 (illustrated in FIG. 1), which supports the patient 14. The couch 82 moves along at least one axis 84 in the x, y, or z directions. In other embodiments of the invention, the patient support can be any device that is adapted to support any portion of the patient's body. The patient support-is not limited to having to support the entire patient's body. The system 10 also can include a drive system 86 operable to manipulate the position of the couch 82. The drive system 86 can be controlled by the computer 74.

The magnetron 32, as shown in FIG. 3, generates microwave radiation that is passed to the LINAC 26. On the highest level, a magnetron takes DC power and converts it to RF power. The power conversion is reciprocal in that the magnetron couples power out in an equal and opposite manner to the power input to the magnetron. One such magnetron that can be utilized according to the present invention is model number MG-6493, supplied by e2v Technologies (UK) LTD. The magnetron 32 is a high power microwave oscillator in which the energy of accelerated electrons emitted from a cylindrical cathode 84 is converted into radio-frequency energy in a series of resonant cavities 88. The resonant cavities 88 are defined by vanes 92. The cathode 84 is surrounded by a concentric anode 104. The magnetron 32 is immersed in a magnetic field applied along the axis of the cathode 84.

As the cathode 84 is heated, electrons are generated that travel radially outwardly, drawn toward the anode 104 by the radial electric field between the cathode 84 and the anode 104. The magnetic field deflects the electrons into curved trajectories between the cathode 88 and the anode 104, inducing RF currents in the cavities 88. This causes energy to be stored in the cavities 88 at the resonant frequency of the cavities 88. The kinetic energy of the electrons is thereby transferred into RF energy, with approximately 60% of the kinetic energy of the electrons getting converted into microwave energy in the illustrated embodiment.

The magnetron 32 of the illustrated embodiment can oscillate in various frequency modes, including a π-mode. To reduce the possibility of oscillations in modes other than the π-mode, the vanes 92 of the magnetron 32 are connected by straps 108. The straps 108 connect alternate vanes 92 that are of equal potential and pass over adjacent vanes 92 which, at π-mode frequency, are 180° out of phase. The RF power is coupled out of the cavities 88 to a circular waveguide section via a coupling loop 120. The coupling is reciprocal, and RF power can be coupled back into the magnetron 32 with the same efficiency as is output by the magnetron 32. A strong coupling increases output power and efficiency but also increases time jitter and sensitivity to changes to load mismatch.

It should be understood that while one particular magnetron configuration is discussed in detail above with respect to FIG. 3, other magnetron configurations are possible and still fall within the scope of the invention. The basic operation and components of a magnetron are well known to those of skill in the art, and one of skill in the art would understand that variations in the magnetron configuration from what are discussed above are possible and still fall within the scope of the invention.

FIG. 4 illustrates the LINAC 26 used in the system 10. The LINAC 26 includes three basic components: an electron gun 128, an accelerator 132, and a target 136. An injector 140 powers the electron gun 128 and injects a pulse of current for each pulse of RF power from magnetron 32. The electron gun 128 includes a cathode that is heated to produce electrons. The electrons produced by the electron gun 128 are drawn toward a gun anode and are injected into the accelerator 132 at about thirteen KV in the illustrated embodiment.

The injected electrons are then grouped into bunches so that a bunch of electrons can be accelerated as one entity by the accelerator 132. The accelerator includes a plurality of accelerating cavities that each includes an applied field that accelerates the electrons as they pass through the cavities. The coupled resonant cavities form a multi-cavity accelerating structure. The number of modes (i.e., the number of operating conditions having a specific resonant frequency and characteristic field pattern) is determined by the number of cavities (i.e., the number of resonators). The accelerator 132 utilized in the illustrated embodiment is a standing-wave accelerator, where electro-magnetic waves get reflected at the ends of the cavities and bounce back and forth, forming a standing wave. However, it is understood that other types of accelerators can be used in the system 10 and still fall within the scope of the invention.

The accelerated electrons are then bombarded against the target 136. The bombardment into the target 136 causes a bremsstrahlung effect. The target 136 slows down the accelerated electrons, causing the emission of X-rays as the deceleration of the electrons occurs. The energy of the emitted X-rays varies with the energy of the bombarding electrons. For example, the emitted X-rays become more energetic and shifts toward higher frequencies when the energy of the bombarding electrons is increased. The target 136 is formed of a high atomic number metal, like tungsten, that can withstand the high heats generated by the bombardment of the electrons. In some cases, a cooling mechanism is utilized by the LINAC to assist in cooling the target 136.

While one particular configuration of the LINAC 26 is described above, one of skill in the art would understand that other LINAC 26 configurations are possible and still fall within the scope of the invention. The LINAC 26 configuration described above is an illustration of one LINAC 26 embodiment for use with the invention. The basic operation and components of a LINAC are understood in the art and one of skill in the art would understand that other LINAC configurations are possible.

As will be discussed in more detail below, the magnetron 32 and LINAC 26 are operatively coupled together such that the magnetron 32 and LINAC 26 work together in the system 10. The magnetron 32 is kept mechanically tuned to the operating frequency of the LINAC 26 by a feedback system, also known as an automatic frequency control 156 (AFC). The AFC 156 drives a motorized plunger (not shown) that perturbs one of the magnetron cavities 88. The plunger acts as the magnetron tuner 158. To minimize frequency deviation when the magnetron 32 is rotated about a horizontal axis, this axis should be parallel to the axis of the tuner 158. The AFC 156 acts as a mechanical tuner and works to tune the frequency by looking at an average of the behavior of the individual RF pulses, and adjusting the tuner 158 in order to minimize power reflected from the LINAC 26. The AFC 156 does not work fast enough to correct an individual RF pulse. Thus, if the magnetron 32 output frequency varies rapidly, such as due to mechanical vibration, the individual pulses can alternate high and low such that the average of the pulses is still within the operating parameters but the magnetron 32 is still operating outside of the desired output frequencies.

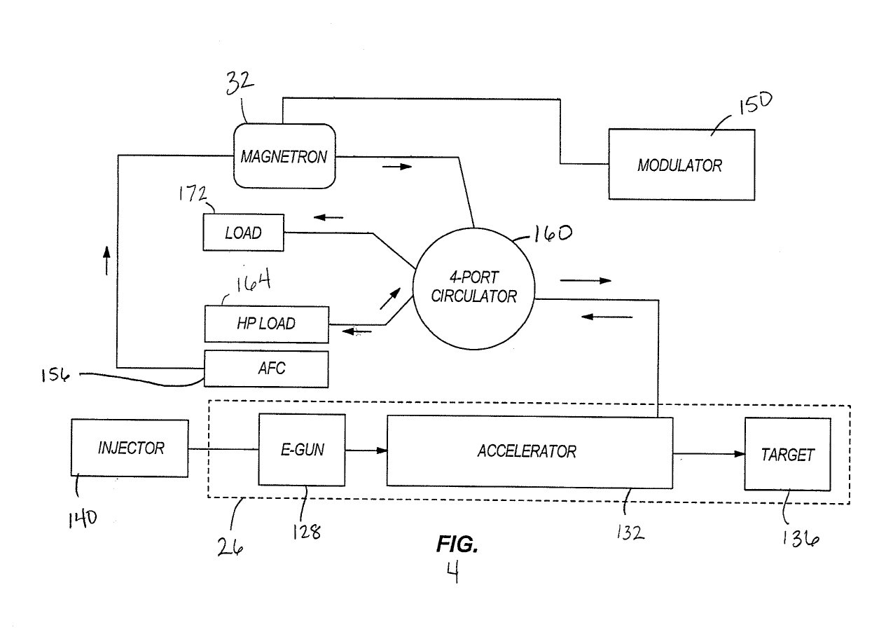

FIGS. 4 and 5 are block diagrams, illustrating the radiation delivery module 22 of the system 10 as a circuit. The magnetron 32 receives power from a modulator 150 that generates short pulses of very high voltage & current. The magnetron 32 passes microwaves through a 4-port circulator 160 to the LINAC 26. The 4-port circulator 160 is a non-reciprocal waveguide device that directs applied power unidirectionally between a sequence of ports. Essentially, the 4-port circulator 160 couples the magnetron 32 to the LINAC 26 and acts as an isolator. The 4-port circulator 160 allows for the independent adjustment of the amplitude and phase of power reflected back from the LINAC 26. It is understood by one of ordinary skill in the art that amplitude and phase are just two characteristics of the energy within the module that could be controlled. In other embodiments, the frequency and the wavelength could also be adjusted.

The reflected power is caused by frequency instability within the circuit. Previous systems utilized a 3-port circulator where a single adjustment, affecting both amplitude and phase, was used. Separation of the phase and amplitude adjustment in the present invention allows both components to be more accurately and easily adjusted, leading to more accuracy in system control and better predictability in operation of the magnetron 32. Separating the amplitude control also allows for simple limiting of the reflected power that reaches the magnetron 32 to insure that the reflected power does not exceed the maximum which the magnetron 32 can tolerate.

The circulator 160 is used to divert power reflected from the LINAC 26 away from the magnetron 32, into a high-power load 164 in order to avoid instability and possible damage to the magnetron 32. As shown in FIG. 4, the circuit includes a fractionally reflecting element (i.e., the reflection transformer 168) in series with the high power load 164. A phase-adjustable, fully reflecting element (i.e., the phase shifter 172) is located on the 4^{th} circulator port between the fractionally reflecting element 168 and the magnetron 32.

By utilizing the 4-port circulator 160 and the components attached thereto, the amplitude and phase of the reflected power (energy) can be separately and independently adjusted. A small amount of reflected power reaches the magnetron 32 when the magnetron 32 is not operating exactly at the LINAC 26 resonant frequency. The action of the reflected power is to modify the frequency of the magnetron 32 in such a way as to eliminate the reflected power, creating a feedback loop. The tuning correction occurs within a fraction of a microsecond at the beginning of each pulse, so in essence there is almost no reflected power reaching the magnetron 32. Thus, the system 10 is electronically tuned to account for the variations of individual RF pulses.

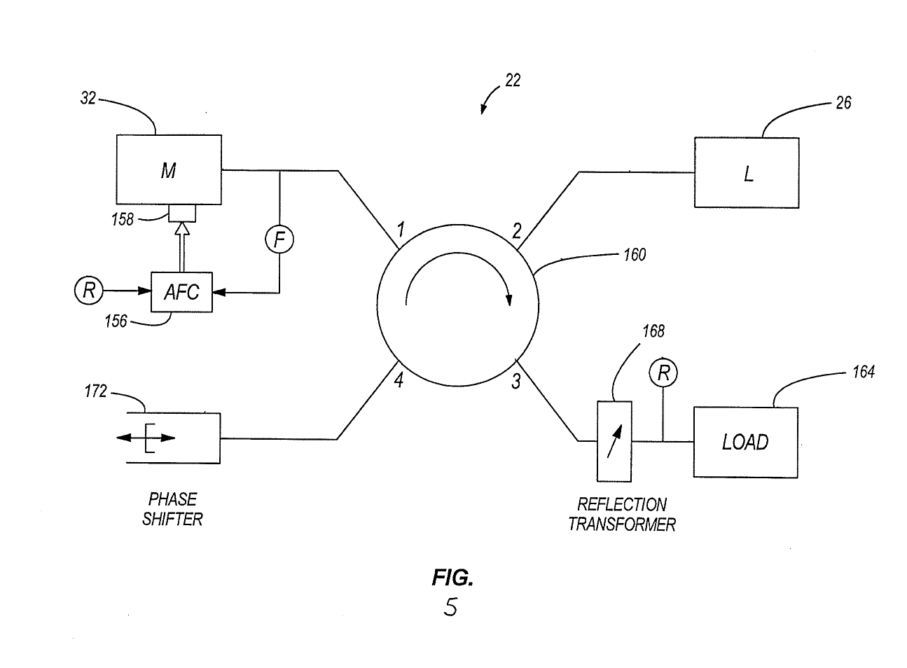

While a 4-port circulator is used in the illustrated embodiment of the invention, it should be understood that other types of devices could be used in place of the illustrated circulator. For example, as illustrated in FIG. 6, a 3-port circulator 180 having separate phase and amplitude controls could be used. In the illustrated 3-port circulator 180, a phase shifter 184, a fractionally reflecting element 188, and a high power load 192 are placed in series coupled to the third port of the 3-port circulator 180. The power reflected from the LINAC 26 is passed through the phase shifter 184 and to the reflecting element 188, where a fraction of the power is reflected. A majority of the power, approx. 98% in some cases, is passed through the reflecting element 188 to the load 192 where the power is dissipated. The reflected power is passed back through the phase shifter 184 and the phase-shifted reflected power is passed to the magnetron 32. The power circulating and port isolating function of the 3-port circulator 180 is essentially the same as the 4-port circulator 160 discussed above—the electrical length of the waveguide is lengthened (to shift the phase) and the phase can be independently controlled, though there may be other benefits to utilizing the 4-port circulator 160 as the coupling mechanism. In addition, any other configuration of a non-reciprocal waveguide device having unidirectional power transfer that allows for independent control of the phase and amplitude of the reflected power within the system could be used according to the invention. For example, a 5-port circulator could be used, as could a circulator having any other number of ports and accomplishing the functions discussed above.

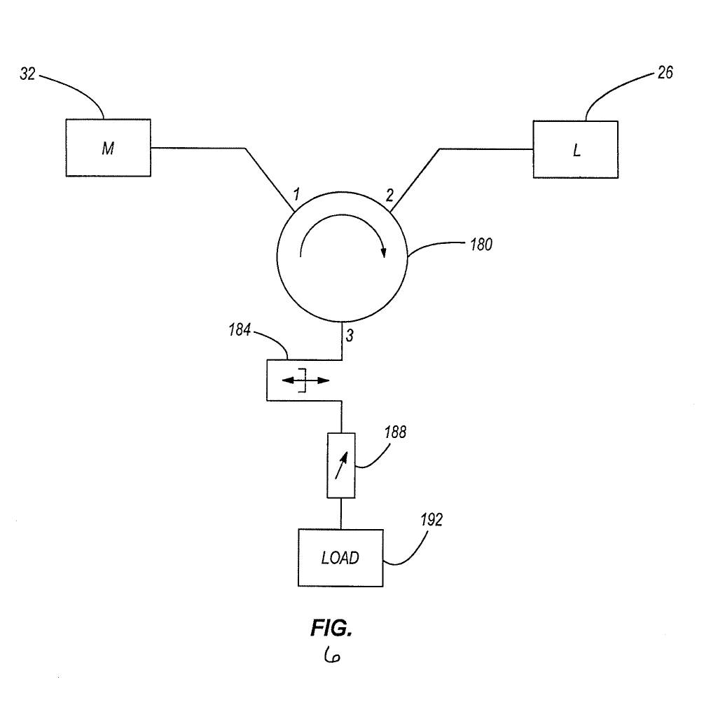

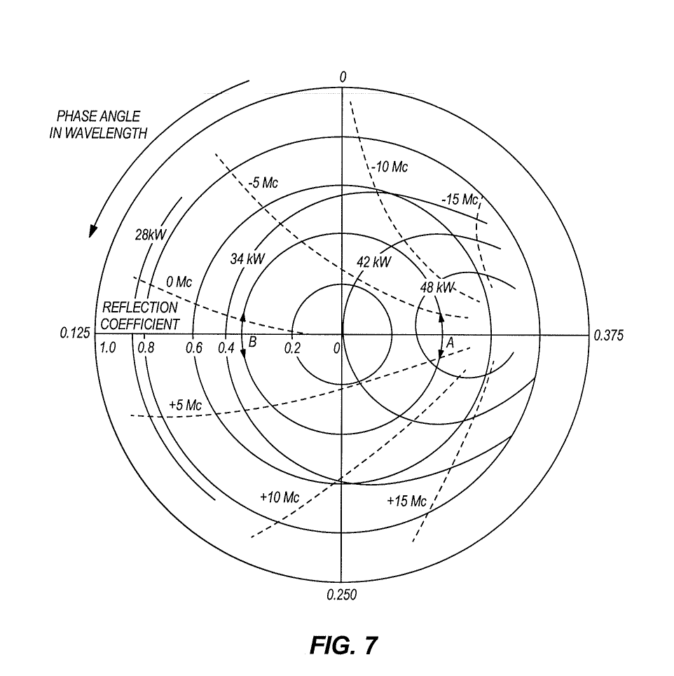

In the illustrated embodiment, 2% of the reflected power is applied to the magnetron 32 in such a phase that the load impedance curve is perpendicular to the equi-frequency curves in a Rieke diagram (see FIG. 7) of the magnetron 32 and pulls the magnetron 32 output frequency toward the resonance frequency of the LINAC 26. When the circuit is properly tuned, the actual reflection is 2% of almost zero. The actual reflected power is chosen to be no greater than the 4% maximum allowed by the design of the magnetron 32, and the minimum reflected power is greater than zero.

FIG. 8 illustrates the LINAC impedance in a complex plane with a center of resonance at fₒₗ and frequency increasing in the clockwise direction, and equifrequency contours in a complex impedance plane, with frequency increasing as illustrated and having a magnetron center frequency fₒₘ. Stable operation should result when fₒₘ=fₒₗ.

FIG. 9 is a graphical representation of how adjustment of the magnetron 32 moves the frequency lines of the Rieke diagram in the complex impedance plane. The magnetron tuner 158 moves the family of equifrequency curves orthogonally to their directions in the impedance plane. This is the purpose of the AFC 156 and is the first adjustable parameter for the impedance and frequency relations. Fundamentally, the magnetron 32 needs to be tuned within the ½ power bandwidth of the LINAC 26, which is the point at which the LINAC impedance becomes tangent to the equifrequency contours (see * - - - * in FIG. 8).

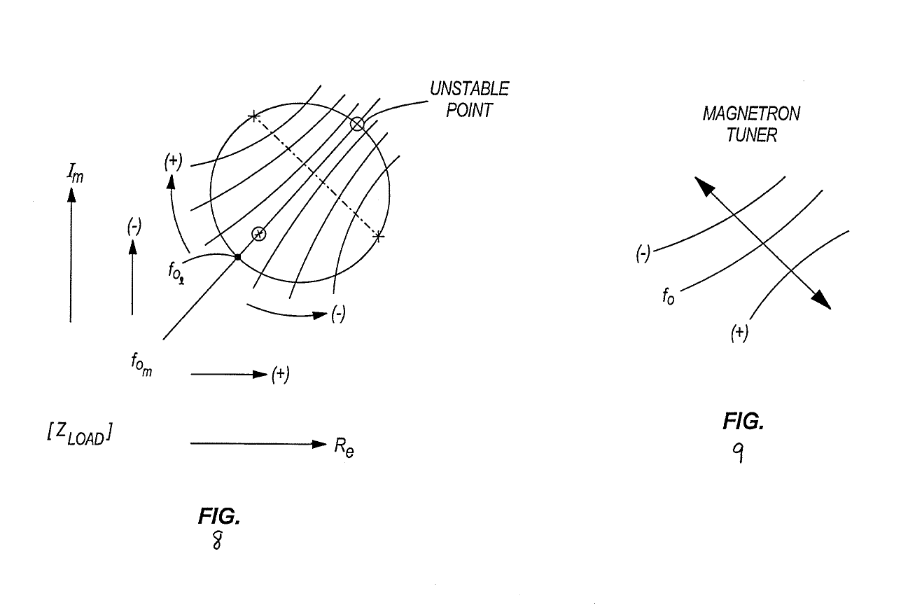

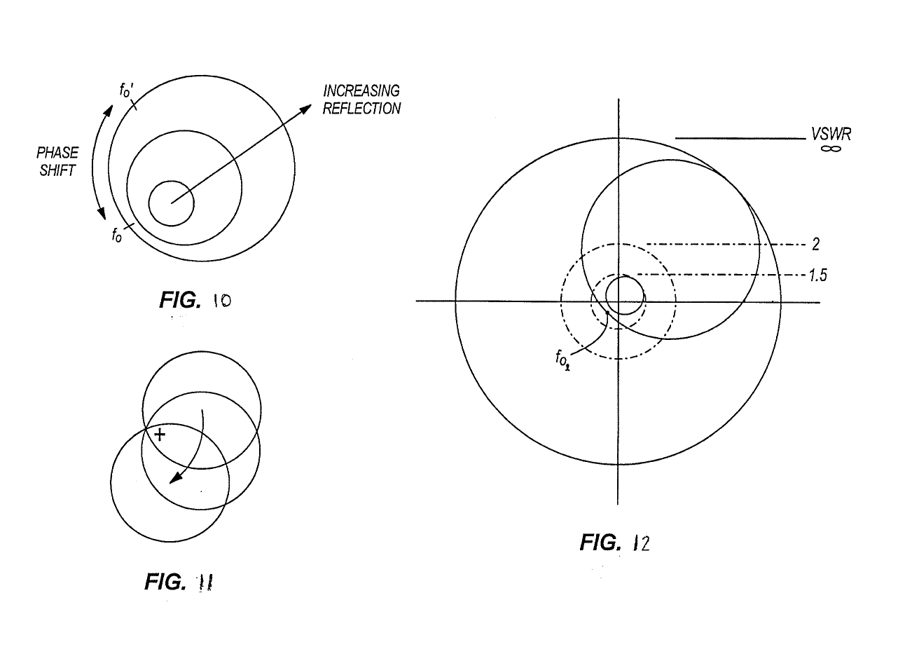

FIG. 10 is a graph displaying how the impedance contour of the LINAC 26 varies with the magnitude of the reflection coefficient at the 3^{rd} port of the 4-port circulator 160. Varying the amounts of reflection added before the high power load 164 changes the magnitude of the LINAC impedance seen by the magnetron 32, as shown graphically in FIG. 10. This is the second adjustable parameter for the impedance and frequency relations.

FIG. 11 is a graph illustrating how the impedance contour of the LINAC 26 varies with the phase of a unit reflection coefficient at the 4^{th} port of the 4-port circulator 160. Varying the phase of a 100% reflection on the usual low power load port (i.e., the 4^{th} port) rotates the impedance curve in the complex impedance plane (see FIG. 11). This is the third adjustable parameter for the impedance and frequency relations.

The second adjustable parameter is controlled by the impedance step in a λ_{g}/4 resonant transformer 168 inserted in series with the high power load 164. The voltage standing wave ratio (VSWR) for such a transformer is:

VSWR=[Z₀/Z₁]².

The general expression for the impedance of a rectangular waveguide operated in the TE₁₀ mode is:

$$Z = \sqrt{\frac{μ}{ɛ} \frac{{λ}_{g}}{{λ}_{0}}} ( \frac{b}{a} )$$

### And where the guide wavelength for vacuum propagation is

$${λ}_{g} = \frac{{λ}_{0}}{\sqrt{1 - {( \frac{{λ}_{0}}{2 a} )}^{2}}}$$

Since λ_{g} does not depend on waveguide height “b”, a reduced height section will have the same frequency dependence as the main guide at a reduced normalized impedance:

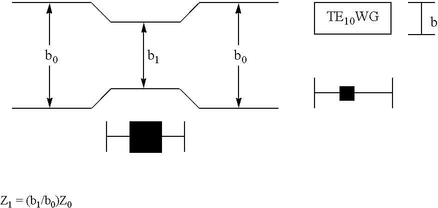

### Thus, the VSWR looking into the transformer is

VSWR=(b₀/b₁)².

### Also, the voltage reflection coefficient Γ=(VSWR−1)/(VSWR+1), and the power reflection coefficient is Γ².

With reference to FIG. 12, in general the LINAC input impedance from one side of resonance to the other presents a circle tangent to the unit circle bounding the Smith chart. FIG. 12 is a graphical representation of how the upper limit of reflected power that can be returned to the magnetron 32 is constrained by the choice of the fractional reflection coefficient at the 3^{rd} port of the 4-port circulator 160. The impedance approaches the origin at resonance, as shown in FIG. 12. This impedance contour is scaled by the reflection coefficient Γ, such that the upper limit of power returned to the magnetron 32 is limited to Γ². For the magnetron 32, the specified upper limit of VSWR is 1.5, which corresponds to a 4% power reflection. When operating within the ½ power bandwidth of the LINAC 26, less than ½ of this power is actually reflected, and on resonance the reflection is dominated by other imperfections in the RF chain.

The third adjustable parameter is controlled by a sliding short 172 (i.e., the phase shifter) installed in place of the usual low power load.

The frequency of the magnetron output can change depending upon the amount of power that is reflected to the magnetron and the phase of the reflected power, and instability in the magnetron 32 output can result from uncontrolled power reflection from the LINAC 26. The use of a circulator essentially isolates the magnetron from the LINAC with respect to reflected power generated by the LINAC. However, the stabilization method described above is utilized to stabilize fluctuations having a totally distinct cause, such as mechanical vibration, to ensure control of the magnetron output. To use the magnetron 32 as the power source to the LINAC 26, the frequency of the output needs to be constrained. In a preferred embodiment, a known, controlled amount of power is reflected back to the magnetron 32 to achieve proper control of the magnetron 32 output.

It should be understood that while the foregoing description describes the invention in relation to its use within a radiation therapy treatment system, that the methods and apparatus described herein for stabilizing a microwave energy source can be used in any other application that requires the stability of the energy output by the microwave energy source. For example, the invention could be applicable to certain microwave radar applications. One of ordinary skill in the art would understand that the specific radiotherapy example discussed in detail herein is just one possible use of the invention and that other uses are possible and still fall within the scope of the invention.

Various features of the invention can be found in the following claims.

I claim:

1. A radiation delivery device comprising:
a microwave energy source;
a microwave utilization device coupled to the energy source; and
a non-reciprocal transmission device that couples the source to the utilization device, the transmission device receiving an unutilized portion of the energy from the utilization device, conditioning at least a portion of the unutilized energy and returning the conditioned energy to the source, the non-reciprocal transmission device comprising a first component that operates to adjust a first property of the energy such that the adjustment does not affect any other properties of the energy;
wherein the returned conditioned energy functions to modify the frequency of the source such that the unutilized energy in the system is minimized.

2. The device of claim 1, wherein the microwave energy source is a magnetron.

3. The device of claim 1, wherein the microwave utilization device is a linear accelerator.

4. The device of claim 1, wherein the non-reciprocal transmission device comprises a circulator.

5. The device of claim 4, wherein the circulator is a 4-port circulator.

6. The device of claim 1, wherein the first component of the transmission device is a fractionally reflecting element.

7. The device of claim 1, wherein the transmission device further comprises a second component that operates to adjust a second property of the energy independent of any other properties of the energy.

8. The device of claim 7, wherein the second component of the transmission device is a phase shifter.

9. The device of claim 1, wherein the first property of the energy that is adjusted by the transmission device includes one of phase, amplitude, wavelength, and frequency.

10. The device of claim 1, wherein the unutilized portion of the energy represents a mismatch between the output frequency of the energy source and the input frequency of the utilization device.

11. The device of claim 1, further comprising a mechanical tuner that operates on the energy source.

12. The device of claim 11, wherein the mechanical tuner adjusts the frequency of the unutilized energy across an average of individual energy pulses.

13. The device of claim 1, wherein the transmission device is capable of conditioning an individual energy pulse such that the transmission tunes the frequency of the energy source on a pulse-by-pulse basis.

14. The device of claim 1, wherein the transmission device is capable of separately adjusting the phase and the amplitude of the unutilized power returned to the energy source.

15. The device of claim 1, wherein the utilization device has a higher frequency stability than the source, and wherein conditioning the unutilized energy stabilizes the frequency output of the source to operate within the frequency bandwidth of the utilization device.

16. A radiation delivery device comprising:
a microwave energy source;
a microwave utilization device, the device constrained to operate within a more precisely controlled frequency bandwidth than provided by the source such that only a portion of the energy from the source can be utilized; and
a non-reciprocal transmission device that receives an unutilized portion of the energy reflected from the utilization device and conditions the unutilized energy prior to returning the conditioned energy to the source;
wherein the transmission device separately and independently adjusts at least two different characteristics of the energy.

17. The device of claim 16, wherein the microwave energy source is a magnetron.

18. The device of claim 16, wherein the microwave utilization device is a linear accelerator.

19. The device of claim 16, wherein the non-reciprocal transmission device comprises a circulator.

20. The device of claim 19, wherein the circulator is a 4-port circulator.

21. The device of claim 16, wherein the conditioned energy acts upon the source to minimize the unutilized energy in the radiation delivery device.

22. The device of claim 16, wherein the transmission device further includes a phase shifter operable to adjust the phase of the energy returned to the source.

23. The device of claim 16, wherein the transmission device further includes a reflecting element operable to adjust the amplitude of the energy returned to the source.

24. The device of claim 16, wherein the transmission device is operable to independently adjust one or more of the following energy characteristics: phase, amplitude, wavelength, and frequency.

25. The device of claim 16, wherein the transmission device separately and independently adjusts the phase and amplitude of the energy.

26. The device of claim 16, wherein the transmission device is capable of conditioning an individual energy pulse such that the transmission device tunes the energy on a pulse-by-pulse basis.

27. A method of stabilizing a microwave energy source, the source coupled to a microwave utilization device that operates within a narrower frequency bandwidth than the source, the method comprising:
coupling a non-reciprocal transmission device to the source;
directing energy from the source through the transmission device to the utilization device;
conditioning at least a portion of the energy returned from the utilization device, the conditioning step including modifying a first characteristic of the energy without affecting any other characteristics of the energy; and
returning the conditioned portion of the energy to the source;
wherein the conditioned energy functions to stabilize the frequency output of the energy source.

28. The method of claim 27, wherein the returned portion of the energy represents a frequency mismatch between the energy source and the utilization device, and wherein returning the conditioned portion of the energy to the source acts to pull the frequency of the source into alignment with the frequency of the utilization device.

29. The method of claim 27, wherein conditioning the portion of the energy includes passing the energy through a phase shifter to modify the energy phase.

30. The method of claim 27, wherein conditioning the portion of the energy includes passing the energy through a reflecting element to modify the energy amplitude.

31. The method of claim 27, wherein conditioning at least a portion of the energy includes conditioning individual pulses of energy output by the source in order to control the frequency of the energy on a pulse-by-pulse basis.
